(19) United States
(12) Patent Application Publication
(10) Pub. No.: US 2009/0170766 A1
(43) Pub. Date: Jul. 2, 2009

(54) Chimeric Kunitz Domains and their Use

(75) Inventors: Felix Oehme, Wuppertal (DE); Heiner Apeler, Concord, CA (US); Frank Dittmer, Düsseldorf (DE); Jürgen Franz, Witten (DE); Axel Harrenga, Wuppertal (DE); Michael Sperzel, Kierspe (DE); Simone Greven, Dormagen (DE); Jürgen Lenz, Leverkusen (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, Leverkusen (DE)

(21) Appl. No.: 12/224,759

(22) PCT Filed: Mar. 1, 2007
(86) PCT No.: PCT/EP2007/001753

§ 371 Date: Jan. 23, 2009

(30) Foreign Application Priority Data
Mar. 8, 2006 (EP) 06004693.5

(51) Int. Cl. A61K 38/16; C07K 14/00; C07H 21/04; C12N 15/63; C12N 1/00; C12P 21/02; A61P 43/00

(52) U.S. Cl. 514/12; 530/324; 536/23.1; 435/320.1; 435/243; 435/69.1

(57) ABSTRACT

The present invention relates to chimeras of human tissue factor inhibitor domain 1 with natural and non-natural Kunitz domains, and their preparation and use.

### RELATED APPLICATIONS/PATENTS AND INCORPORATION BY REFERENCE

This application is a National Stage Application filed under 35 U.S.C. § 371 based on International Application No. PCT/EP2007/001753, filed Mar. 1, 2007, which claims priority to European Patent Application Number 06004693.5, filed Mar. 8, 2006, the entire contents each of which are incorporated herein by reference.

The foregoing applications, and all documents cited therein and all documents cited or referenced therein, and all documents cited or referenced herein, including any U.S. or foreign patents or published patent applications, International patent applications, as well as, any non-patent literature references and any manufacturer's instructions, are hereby expressly incorporated herein by reference.

### BACKGROUND

1. Field of the Invention

The present invention relates to chimeras of human tissue factor inhibitor domain 1 with natural and non-natural Kunitz domains, and their preparation and use.

2. Background of the Invention

Kunitz domains are polypeptides which inhibit a large number of serine proteases of varying potency. They comprise three disulphide bridges which stabilize the protein and determine its three-dimensional structure. Interaction with the respective serine protease takes place principally via a loop which is about 9 amino acids long and is in the N-terminal region of the Kunitz domain. This loop binds to the catalytic centre of the protease and thus prevents cleavage of the appropriate protease substrates (Laskowski and Kato [1980]; Bode and Huber [1992]).

Aprotinin (BPTI; FIG. 1; SEQ ID NO: 6) is regarded as prototype of Kunitz domains (Fritz and Wunderer [1983]). This is a basic protein with a length of 58 amino acids which can be isolated from various bovine organs (inter alia pancreas, lung, liver and heart). Aprotinin is stabilized by three disulphide bridges (Cys 5-Cys 55; Cys 14-Cys 38; Cys 30-Cys 51) and is inter alia a potent inhibitor of trypsin, plasmin and plasma kallikrein.

It has been possible to show by X-ray structural analysis of the complex between aprotinin and bovine trypsin that the contact region of aprotinin with the catalytic centre of the protease is formed essentially by a loop consisting of amino acids 11 to 19 (see Bode and Huber [1992] and references listed therein). The amino acid Lys 15 is of central importance for the inhibitory effect of aprotinin and is in particularly close contact with the catalytically active serine residue of the protease. The amino acid Lys 15 is therefore referred to by definition as P1 residue (Schechter and Berger [1967]. The P2, P3 etc. residues are located N-terminally of Lys-15, while the amino acids located C-terminally from Lys-15 are referred to as P1′, P2′ etc. Since Lys-15 is located directly C-terminally next to the second cysteine residue of aprotinin, the corresponding amino acid in this position in other Kunitz domains is likewise referred to as the P1 residue. It has been shown earlier that the inhibitory effect of aprotinin can be modified by targeted exchange of amino acids in the region from residue 11 to residue 19 (Otlewski et al. [2001], Apeler et al. [2004], Krowarsch et al. [2005]). The amino acids 36-39 are additionally important for the activity of aprotinin (Fritz and Wunder [1983], Krowarsch et al. [2005]).

Aprotinin is now mainly employed under the proprietary name Trasylol in cardiac surgery since clinical studies have shown that treatment with aprotinin significantly reduces the need for transfusion in such operations and leads to a reduction in secondary haemorrhages (Royston [1992]). Its clinical effect is attributed to the inhibition of the intrinsic coagulation of blood (contact activation), the inhibition of fibrinolysis and the reduction in thrombin formation (Blauhut et al. [1991], Dietrich et al. [1995]). Thus, the inhibition both of plasmin and of plasma kallikrein is important for the haemostatic effect of aprotinin.

Aprotinin as bovine protein leads to the formation of antibodies in humans. Repeated administration of Trasylol may lead to severe allergic reactions (anaphylactic shock). The risk of this is 2.8%, and thus the possibility of multiple use of aprotinin is greatly restricted (Dietrich et al. [2001], Beierlein et al. [2005]). There is thus a great medical demand for active ingredients which have a similar or better clinical effect than aprotinin and which elicit significantly less of an allergic reaction.

Activation of the coagulation of blood by the extrinsic pathway (e.g. in tissue injuries) is initiated by release of tissue factor from endothelial cells (Lindahl [1997], Lwaleed and Bass [2005]). When tissue factor enters the blood it binds to factor VIIa. The complex formed thereby activates both factor X (extrinsic coagulation of blood) and factor IX (intrinsic coagulation of blood). Under physiological conditions, tissue factor is inhibited by the tissue factor inhibitor (TFPI). Human tissue factor inhibitor (hTFPI) is a protein with a length of 276 amino acids and a molecular mass of about 42 kDa. It inhibits firstly factor Xa and then binds to the factor VIIa/tissue factor complex. TFPI consists of three Kunitz domains and, in plasma, is mainly bound to lipoproteins. It is therefore also referred to as “lipoprotein-associated coagulation inhibitor” (LACI). TFPI was purified for the first time from supernatants from human HepG2 cells (Broze et al. [1987]) and a little later from human plasma (Novotny et al. [1989]). The cDNA of hTFPI was cloned in 1988 (Wun et al. [1988]). Sprecher et al. describe the cloning and characterization of a further isoform of the tissue factor inhibitor (hTFPI2; Sprecher et al. [1994]).

It was possible to show by targeted exchange of the amino acids in the P1 position of the respective Kunitz domains of hTFPI that Kunitz domain 2 (D2) is responsible for the inhibition of factor Xa, while Kunitz domains 1 (D1) and 2 are necessary for binding to the factor VIIa/tissue factor complex (Girard et al. [1989]). Investigations with separately expressed Kunitz domains of hTFPI have shown that factor Xa can be inhibited only by Kunitz domain 2, whereas Kunitz domain 1 also inhibits plasmin (Petersen et al. [1996]). Markland et al. describe the preparation of protein libraries by variation of hTFPI D1 by means of phage display. They were able to modify, through mutations in the region of amino acids 10 to 21 and 31 to 39, the properties of hTFPI D1 in such a way that the Kunitz domains resulting therefrom are either highly potent, selective plasmin inhibitors (Markland et al. [1996a]) or highly potent, selective inhibitors of plasma kallikrein (Markland et al. [1996b]). Baja et al. discuss the significance of amino acids 8 to 20 and 31 to 39 for the inhibitory activity of hTFPI D1 (Bajaj et al. [2001]).

Variants of Kunitz domains which inhibit plasmin with high potency and specifically are described in U.S. Pat. No. 6,010,880; EP 0 737 207; U.S. Pat. No. 6,071,723 and U.S. Pat. No. 6,953,674. U.S. Pat. No. 5,795,865; EP 0 739 355 and US2004/0038893 disclose variants of Kunitz domains which inhibit plasma kallikrein with high potency and specifically. U.S. Pat. No. 6,783,960 describes chimeric proteins consisting of various Kunitz domains of hTFPI1 and/or hTFPI2. Plasma kallikrein inhibitors of the Kunitz type are disclosed in U.S. Pat. No. 5,786,328; U.S. Pat. No. 5,780,265 and EP 0 832 232.

### SUMMARY OF THE INVENTION

The aim of the present invention is to prepare variants of human tissue factor inhibitor 1 domain 1 (hTFPI D1) with an activity which is comparable to or better than that of aprotinin while having significantly less immunogenicity. The desired properties are achieved by exchanging amino acids in the active centre of hTFPI D1 for corresponding amino acids from the active centre of other natural or non-natural Kunitz domains. The chimeras thus generated inhibit both plasmin and plasma kallikrein with high potency.

### BRIEF DESCRIPTION OF THE FIGURES

The following detailed description, given by way of example, but not intended to limit the invention solely to the specific embodiments described, may best be understood in conjunction with the accompanying drawings, in which:

FIG. 1 shows the amino acid sequence of some preferred variants according to the invention of hTFPI D1, also the sequences of aprotinin (BPTI) and hTFPI D1.

FIG. 2 shows the effect of Trasylol (aprotinin) and of the variant according to the invention TFPI mut3EA (SEQ ID NO: 19) on fibrinolysis in human plasma (in vitro).

FIG. 3 shows the effect of Trasylol (aprotinin) and of the variant according to the invention TFPI mut3 EA (SEQ ID NO: 19) on coagulation in human plasma (in vitro).

FIG. 4 shows the effect of Trasylol (aprotinin) and of the variant according to the invention TFPI mut3EA (SEQ ID NO: 19) on the bleeding time in the rat in the presence of TPA (in vivo).

FIG. 5 shows the nucleotide sequence and derived amino acid sequence of the synthetic hTFPI D1 domain (hTFPI D1, in bold), cloned in the vector pIU10.10.W. Recognition sequences of the restriction enzymes (BsaBI, XhoI) used for the subcloning are underlined.

FIG. 6 shows the nucleotide sequence and derived amino acid sequence of the synthetic hTFPI D1 domain (hTFPI D1, in bold), cloned in the vector pIU3.12.M. Recognition sequences of the restriction enzymes (HindIII, BamHI) used for the subcloning are underlined, and PCR primers employed have a grey background.

FIG. 7 shows the effect of Trasylol (aprotinin) and of the TFPI mut4EA variant according to the invention (SEQ ID NO: 20) on the fibrinolysis in human plasma (in vitro).

FIG. 8 shows the effect of Trasylol (aprotinin) and of the TFPI mut4EA variant according to the invention (SEQ ID NO: 20) on the coagulation in human plasma (in vitro).

FIG. 9 shows the effect of Trasylol (aprotinin) and of the TFPI mut4EA variant according to the invention (SEQ ID NO: 20) on the bleeding time in the rat in the presence of TPA (in vivo).

FIG. 10 shows the antithrombotic effect of Trasylol (aprotinin) and of the TFPI mut4EA variant according to the invention in a rat arteriovenous (AV) shunt model.

FIG. 11 shows the effect of Trasylol on the relative mesenteric bleeding time in rats. The bleeding time after administration of the test substance was related to the average control bleeding time before administration of the substance. The values have been shown as mean±SEM, n=8.

* significantly different from vehicle, P<0.05; *** significantly different from vehicle, P<0.001.

FIG. 12 shows the effect of the TFPI mut4EA variant according to the invention on the relative mesenteric bleeding time in rats. The bleeding time after administration of the test substance was related to the average control bleeding time before administration of the substance. The values have been shown as mean±SEM, n=8.

** significantly different from vehicle, P<0.01; *** significantly different from vehicle, P<0.001.

FIG. 13 shows the inhibition of the IL-8-induced chemotaxis of neutrophils by the TFPI mut4EA variant according to the invention.

FIG. 14 shows the inhibition of the CAP-37-induced increase in permeability by the TFPI mut4EA variant according to the invention

### DETAILED DESCRIPTION OF THE INVENTION

Kunitz domains in the context of this invention are homologues of aprotinin having 55 to 62 amino acids which comprise six cysteine residues and three disulphide bridges. The amino acids are, as shown in Table 1, numbered in accordance with the 58 amino acids of aprotinin. Thus, Cys1 is amino acid 5, Cys 6 is amino acid 55. In Table 1, “x” means any amino acid and “X” means one of the amino acids in each case specified in detail. Disulphide bridges are formed in each case between the positions Cys 5-Cys 55; Cys 14-Cys 38 and Cys 30-Cys 51.

Kunitz inhibitors have to date been found inter alia in various vertebrates (e.g. human, cattle) and in some invertebrates (slug, sea anemone) (Laskowski and Kato [1980] and references cited therein). Such naturally occurring Kunitz domains are referred to as “natural Kunitz domains” in this application. Examples of natural Kunitz domains are inter alia aprotinin, human placental bikunin domain 1, human placental bikunin domain 2, the Kunitz domain of the human amyloid beta A4 protein precursor and the Kunitz domain of the human Eppin precursor. The sequences of some natural Kunitz domains are detailed in Table 2.

It is possible by means of genetic engineering methods to prepare recombinant variants of Kunitz domains which differ from natural Kunitz domains in one or more amino acids. Such Kunitz domains resulting from exchange of amino acids are referred to as “non-natural Kunitz domains” in this application. Various non-natural Kunitz domains have been described in the literature (Dennis et al. [1995], Markland et al. [1996a], Markland et al. [1996b], Apeler et al. [2004], EP 0307592). The sequences of some non-natural Kunitz domains are shown in Table 3.

The aim of this invention is to prepare a Kunitz domain which has a clinical effect which is comparable to or better than that of aprotinin but which elicits significantly less of an allergic reaction in humans than aprotinin. hTFPI D1 is a human Kunitz domain having the sequence:

(SEQ ID NO: 2)

MHSFC AFKAD DGPCK AIMKR FFFNI FTRQC EEFIY GGCEG
NQNRF ESLEE CKKMC TRD

Because of the human origin, no allergic reaction comparable to that of aprotinin is to be expected on use of hTFPI D1 in humans.

Aprotinin is a potent inhibitor of plasmin and plasma kallikrein (Table 4) whose clinical effect is attributed to the intrinsic coagulation of blood (contact activation), the inhibition of fibrinolysis and the reduction in thrombin formation (Blauhut et al., Dietrich et al. [1995]). In contrast thereto, hTFPI D1 is a considerably weaker inhibitor of plasmin and moreover shows no inhibitory effect on plasma kallikrein (Table 4). The aim of this invention is therefore to prepare variants of hTFPI D1 with minimal immunogenic potential and which additionally inhibit both plasmin and plasma kallikrein with high potency. Preferred variants according to the invention inhibit plasmin with an IC50 of <100 nM, but better with an IC50 of <10 nM. Plasma kallikrein is inhibited by preferred variants according to the invention with an IC50<100 nM, but better with an IC50 of <10 nM.

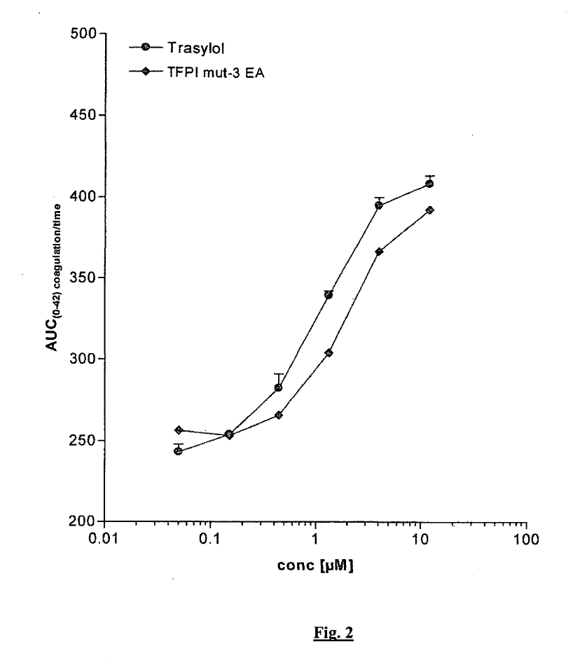

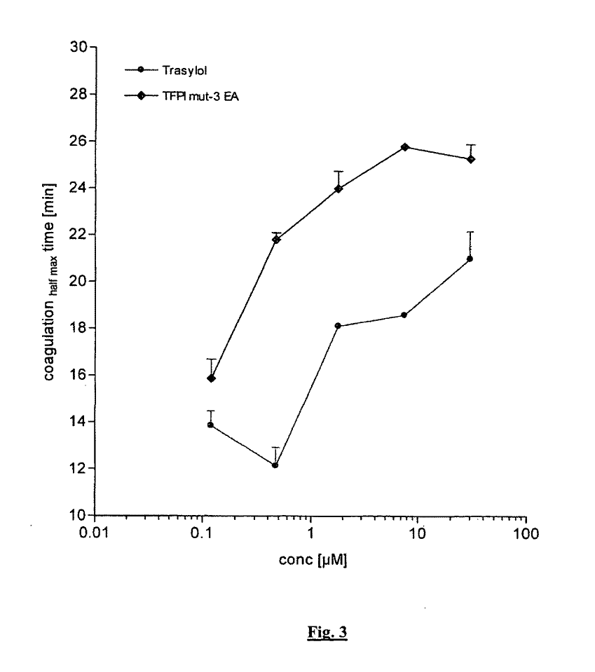

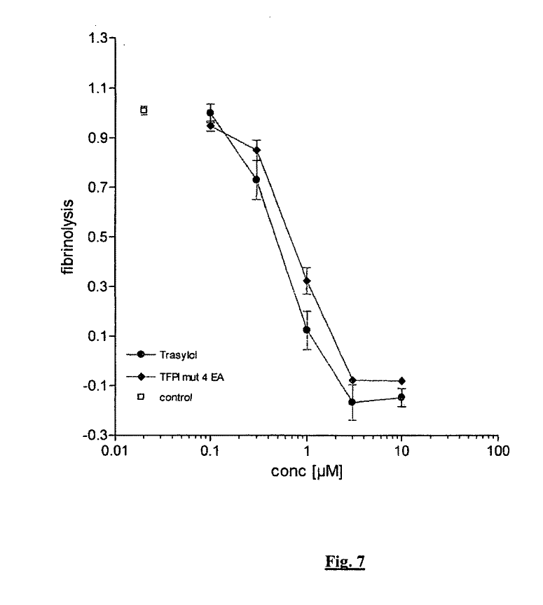

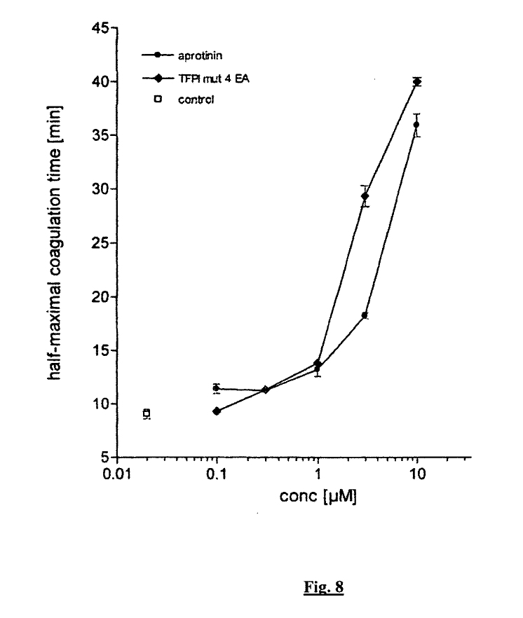

Variants according to the invention of hTFPI D1 are produced by forming chimeras between hTFPI D1 and other natural or non-natural Kunitz domains. Formation of the chimeras takes place by exchanging one or more amino acids in the active centre of hTFPI D1 for corresponding amino acids from the active centre of other natural or non-natural Kunitz domains. The active centre includes amino acids 10 to 21 and 31 to 39 of the corresponding Kunitz domain. It has surprisingly emerged that variants of hTFPI D1 produced thereby exhibit properties which are not only comparable to those of aprotinin but in some cases are distinctly better. Thus, it has emerged for example that TFPI variants according to the invention have a substantially stronger inhibitory effect on plasma kallikrein than aprotinin (Table 4). In addition, TFPI variants according to the invention have been found to have a similar or distinctly improved anticoagulant effect compared with aprotinin (FIG. 3, FIG. 8). Fibrinolysis in human plasma was inhibited by TFPI variants according to the invention with a potency comparable to that of aprotinin (FIG. 2, FIG. 7).

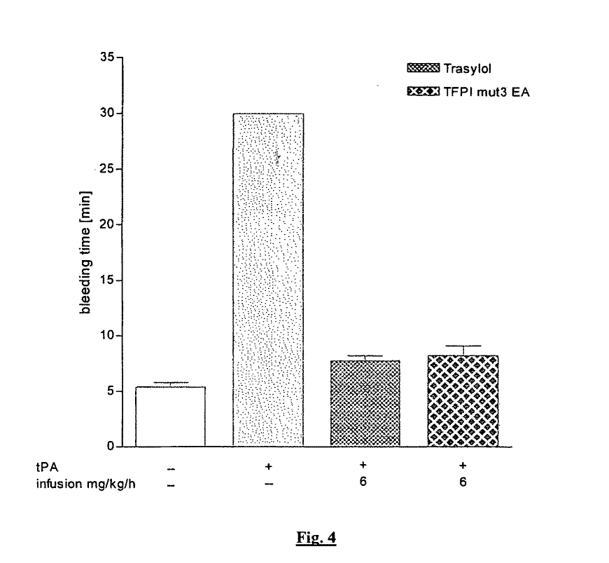

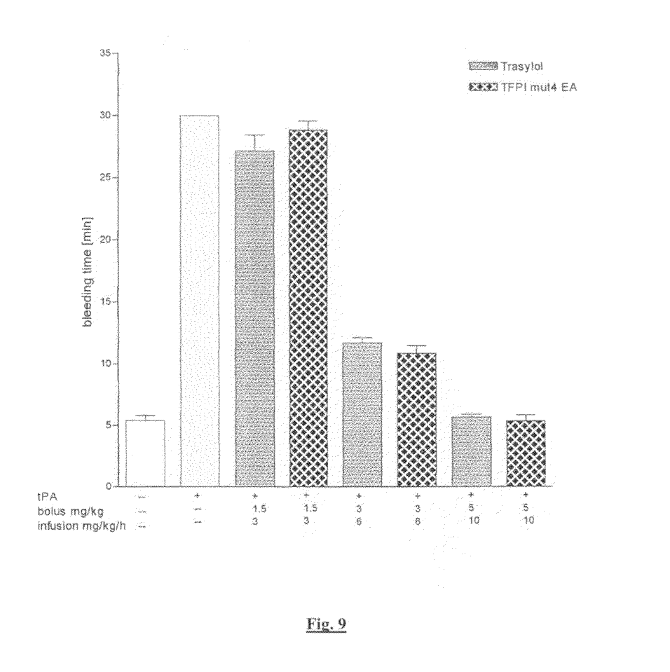

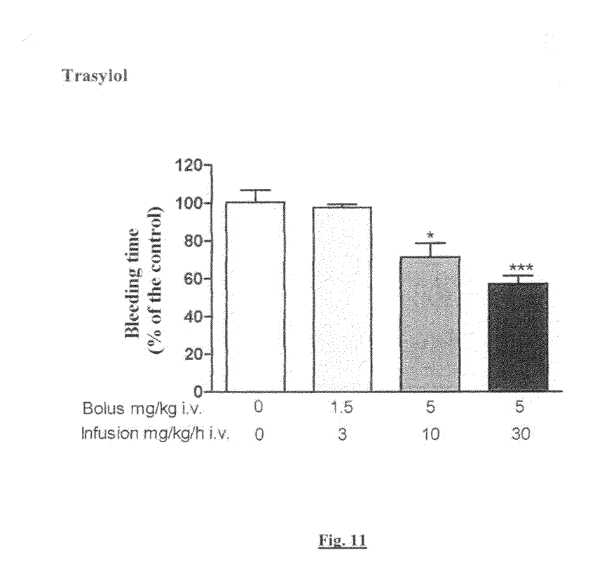

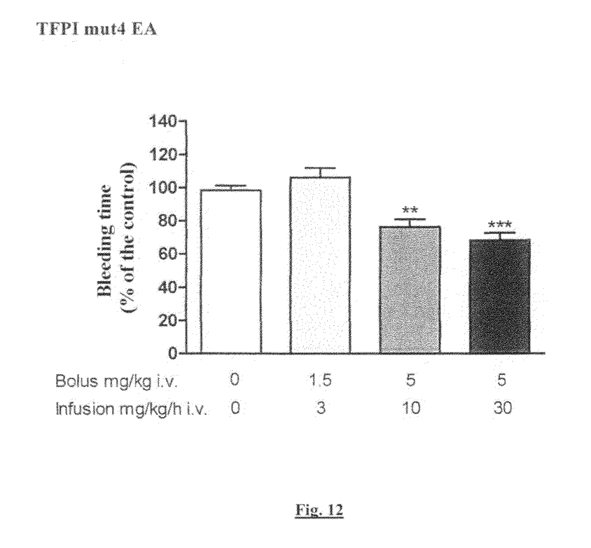

The haemostatic effect of TFPI variants according to the invention was tested in various animal models. In a rat bleeding model, TFPI variants according to the invention showed an effect comparable to that of aprotinin (FIG. 4, FIG. 9). The rat mesenteric bleeding time was reduced by aprotinin and TFPI variants according to the invention with comparable potency (FIG. 11, FIG. 12).

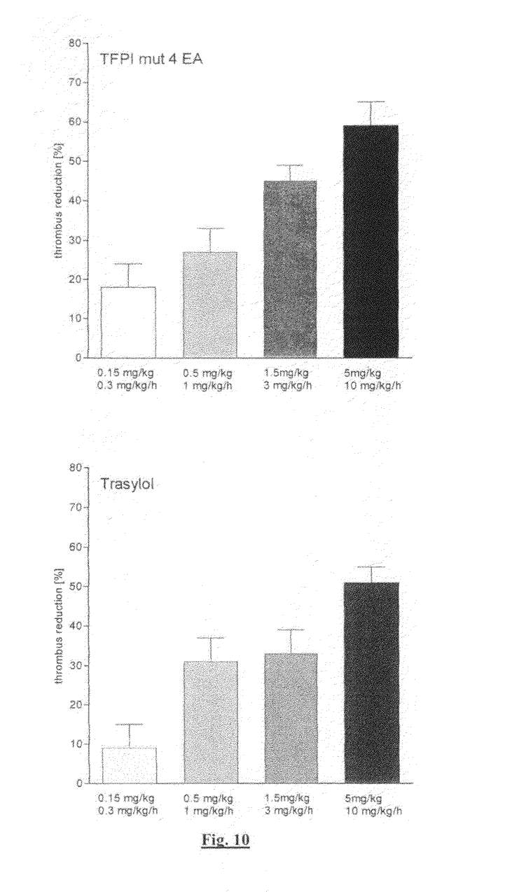

The antithrombotic effect of TFPI variants according to the invention was investigated in a rat arteriovenous shunt model. In this case, TFPI variants according to the invention showed an antithrombotic effect comparable to that of aprotinin (FIG. 10).

Possible unwanted effects of TFPI variants according to the invention on the cardiovascular system were examined in anaesthetized rats. TFPI variants according to the invention showed no effect on blood pressure and ECG on intravenous administration of a dose of up to 50 mg/kg (Table 8).

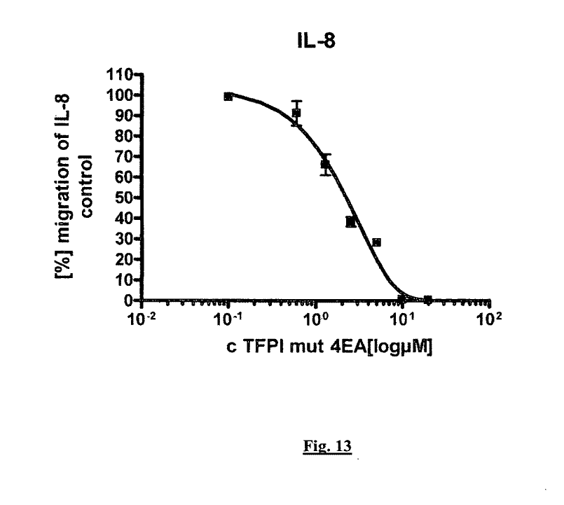

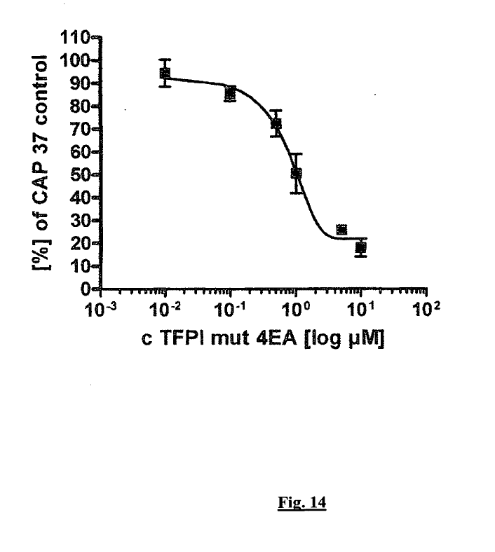

Trauma, reperfusion and extracorporeal circulation are causes of complex inflammatory processes, with activation of humoral and cellular cascade systems. These lead to activation of, inter alia, leukocytes and platelets which cause the clinically observable side effects such as oedema formation and organ damage (Hess P J Jr. [2005]). Various studies show possible effects of aprotinin on the inflammatory status of bypass patients (Asimakopoulos G. et al. [2000]). The possible influence of TFPI variants according to the invention on inflammatory processes was tested in various cellular test systems. Thus, TFPI variants according to the invention inhibited the IL-8- and C5a-induced chemotaxis of neutrophils with a potency which is comparable to or slightly better than that of aprotinin (FIG. 13, Table 6). The CAP-37-induced increase in permeability was likewise inhibited by TFPI variants according to the invention with a potency comparable to or slightly better than that of aprotinin (FIG. 14, Table 7).

Variants according to the invention of hTFPI D1 have the general formula:

(SEQ ID NO: 32)

MHSFC AFKAX₁₀X₁₁GPCX₁₅X₁₆X₁₇X₁₈X₁₉X₂₀X₂₁X₂₂FNI
FTRQC EEFIY GGCEG NQNRF ESLEE CKKMC TRD
or

(SEQ ID NO: 33)

EAMHSFCAFKAX₁₀X₁₁GPCX₁₅X₁₆X₁₇X₁₈X₁₉X₂₀X₂₁X₂₂FNIFTR
QCEEFIYGGCEGNQNRFESLEECKKMCTRD.

In this, “X” is an amino acid of a Kunitz domain according to the numbering shown in Table 1. This amino acid may be derived either from hTFPI D1 or from another natural or non-natural Kunitz domains detailed in Tables 2 and 3. Variants according to the invention are produced by exchange of at least one amino acid of hTFPI D1 for another amino acid of the corresponding natural or non-natural Kunitz domain. Amino acids from natural and non-natural Kunitz domains which can preferably be exchanged for the corresponding amino acids from hTFPI D1 are summarized by way of example in Table 5.

The following variants of hTFPI D1 are particularly preferred:

(SEQ ID NO: 4)

MHSFC AFKAE TGPCRA AHPRWF FNIFT RQCEE FIYGG CEGNQ
NRFES LEECK KMCTR D

(SEQ ID NO: 5)

MSSFC AFKAE TGPCR AAHPR WWFNI FTRQC EEFIY GGCEG
NQNRF ESLEE CKKMC TRD

(SEQ ID NO: 19)

EAMHS FCAFK AETGP CRAAH PRWFF NIFTR QCEEF IYGGC
EGNQN RFESL EECKK MCTRD

(SEQ ID NO: 20)

EAMHS FCAFK AETGP CRAAH PRWWF NIFTR QCEEF IYGGC
EGNQN RFESL EECKK MCTRD

The IC50 values of some hTFPI D1 variants according to the invention for the inhibition of trypsin, plasmin and plasma kallikrein are summarized in Table 4.

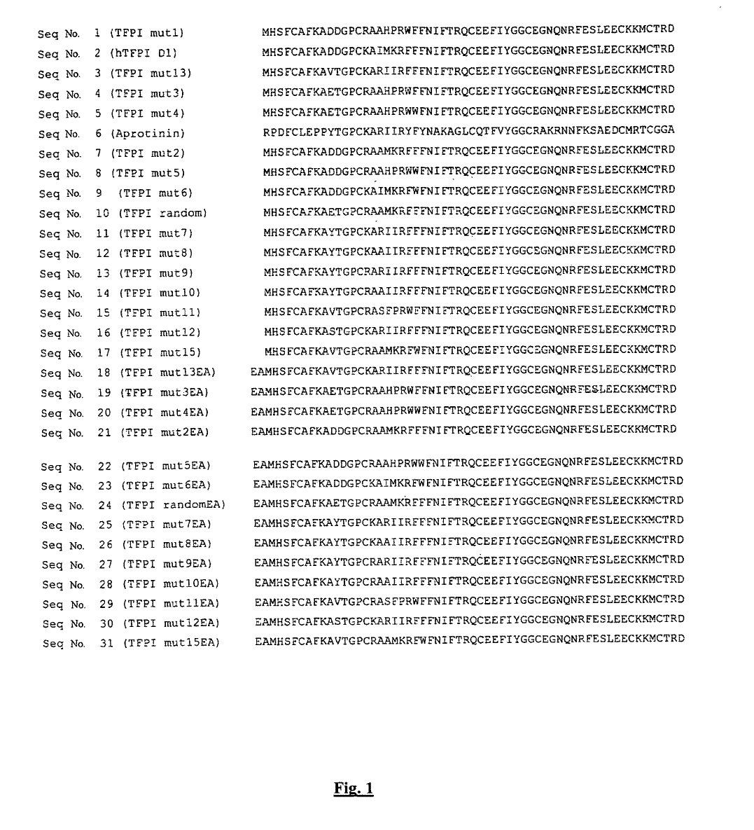

Further variants according to the invention of hTFPI D1 are shown in FIG. 1.

This invention also relates to medicaments which comprise one or more of the variants according to the invention of hTFPI D1.

The described novel Kunitz domains are suitable for the treatment of the following pathological states:

blood loss associated with operations with an increased risk of haemorrhage; therapy of thromboembolic conditions (e.g. after operations, accidents), shock, polytrauma, sepsis, disseminated intravascular coagulation (DIC), multiorgan failure, unstable angina, myocardial infarction, stroke, embolism, deep vein thromboses, inflammatory disorders (e.g. rheumatism, asthma), invasive tumour growth and metastasis, therapy of pain and oedema (cerebral oedema, spinal cord oedema), prevention of activation of haemostasis in dialysis treatment, treatment of symptoms of skin ageing (elastosis, atrophy, wrinkling, vascular alterations, pigment alterations, actinic keratosis, blackheads, cysts), skin cancer, treatment of symptoms of skin cancer (actinic keratosis, basal cell carcinoma, squamous cell carcinoma, malignant melanoma), multiple sclerosis, fibrosis, cerebral haemorrhage, inflammations of the brain and spinal cord, infections of the brain.

### EXAMPLES

### Example 1

### Cloning of the Human Tissue Factor Inhibitor Domain 1 (hTFPI, Tissue Factor Pathway Inhibitor) and Generation of TFPI Variants

The commercially available E. coli/S. cerevisiae shuttle vector pYES2 (Invitrogen) was modified (see Apeler [2005]) and served as starting material for construction of the yeast secretion vectors pIU10.10W and pIU3.12.M.

pIU10.10.W

MFa1 promoter—MFa1-Met1-Arg2 . . . presequence . . . Ala17-Leu18-Ala19|signal peptidase

pIU3.12.M

MFa1 promoter—MFa1-Met1-Arg2 . . . preprosequence . . . Asp83-Lys84-Arg85|KexII protease

The naturally occurring domain 1 of human TFPI (hTFPI D1, acc. P10646, amino acid Met49-Asp107, here: Met1-Asp58) was fused as synthetic gene (optimized for S. cerevisiae codon usage) either to Ala19 in the yeast secretion vector pIU10.10.W (via the restriction cleavage sites BsaBI and XhoI) or to Arg85 in the yeast secretion vector pIU3.12.M (by means of PCR and the restriction cleavage sites HindIII and BamHI).

Cloning of hTFPI D1 into Yeast Secretion Vector pIU10.10.W

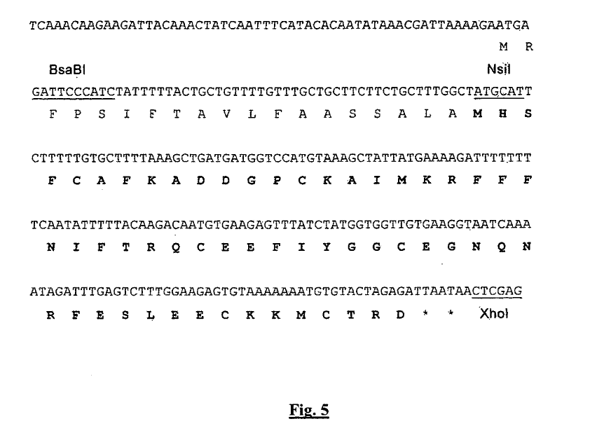

The synthetically prepared gene for hTFPI D1 (Seq No. 2) was subcloned by means of the restriction enzyme BsaBI (5′) and XhoI (3′) into the yeast secretion vector pIU10.10.W (FIG. 5) and the nucleotide sequence was checked by DNA sequence analysis. The preparation of hTFPI D1 variants in yeast by transformation of pIU10.10.W leads to the expression of hTFPI D1 variants having the N-terminal amino acid sequence MHSF.

Cloning of hTFPI D1 into Yeast Secretion Vector pIU3.12.M

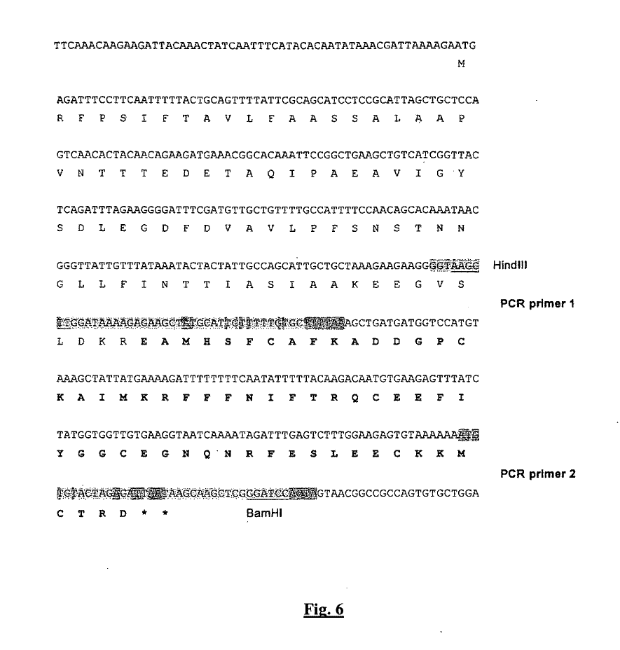

hTFPI D1 (Seq No. 2) was subcloned by means of the polymerase chain reaction (PCR) and appropriate PCR primers with suitable restriction cleavage sites (PCR primer 1/HindIII, 5′ and PCR primer 2/BamHI, 3′) into the yeast secretion vector pIU3.12.M (FIG. 6) and the nucleotide sequence was checked by DNA sequence analysis.

The following PCR primers 1 and 2 were used for subcloning of hTFPI D1 into the yeast secretion vector pIU3.12.M. Recognition sequences for the restriction enzymes (HindIII, BamHI) used for the subcloning are underlined.

PCR primer 1:

(SEQ ID NO: 34)

HindIII
5′-GGTA AGCT TGGA TAAA AGAG AAGC TATG CATT TTTT
TTGT GCTT TTAA-3′
PCR primer 2:

(SEQ ID NO: 35)

BamHI
5′-TAGT GGAT CCCG AGCT TGCT TATT AATC TCTA GTAC
ACAT T-3′

The preparation of hTFPI D1 variants in yeast by transformation of pIU3.12.M leads to the expression of hTFPI D1 variants having the N-terminal amino acid sequence EAMHSF.

Generation of hTFPI D1 Variants
Variant 1: TFPI mut1 (SEQ ID NO: 1)

TFPI mut1 has the following amino acid exchanges by comparison with hTFPI D1: K15R, I17A, M18H, K19P, F21W

TFPI mut1 was generated by means of PCR and appropriately modified PCR primers (PCR primers 3 and 4).

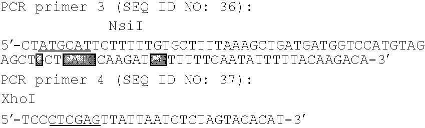

Nucleotide exchanges leading to corresponding amino acid exchanges are shown in bold with a grey background in PCR primer 3.

The DNA fragment amplified with PCR primers 3 and 4 was cloned via the restriction cleavage sites NsiI (5′) and XhoI (3′) into the yeast secretion vector pIU10.10.W. The PCR primers described in section 2 (PCR primer 1 and PCR primer 2) were used for subcloning TFPI mut1 into the yeast secretion vector PIU3.12.M. The nucleotide sequences were checked in each case by DNA sequence analyses.

Variant 2: TFPI mut3 (SEQ ID NO: 4)

TFPI mut3 has by comparison with hTFPI D1 and TFPI mut1 further amino acid exchanges: D10E, D11T, K15R, I17A, M18H, K19P, F21W (SEQ ID NO: 4)

TFPI mut3 was generated by in vitro mutagenesis (using the Quick-change II XL site-directed mutagenesis kit, from Stratagene), starting from TFPI mut1 using the mutagenesis primers 1 and 2

The initial sequence in TFPI mut1 (partial sequence) to be mutated is (SEQ ID NO: 38):

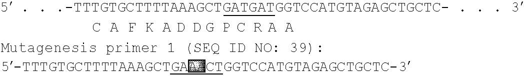

Mutagenesis primer 1 comprises instead of the triplets GAT/D10 and GAT/D11 the modified triplets

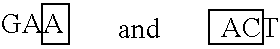

and thus leads to the amino acid exchanges D10E and D11T.

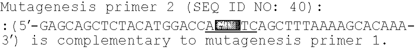

The mutagenesis was carried out in accordance with the manufacturer's information, and the nucleotide sequence was then checked by DNA sequence analysis.

### Example 2

### Transformation of Saccharomyces cerevisiae

Yeast cells e.g. of the strain JC34.4D (MAT□, ura3-52, suc2) were grown in 10 ml of YEPD (2% glucose; 2% peptone; 1% Difco yeast extract) and harvested at an OD_{600 nm} of 0.6 to 0.8. The cells were washed with 5 ml of solution A (1 M sorbitol; 10 mM bicine pH 8.35; 3% ethylene glycol), resuspended in 0.2 ml of solution A and stored at −70° C.

Plasmid DNA which comprises the gene coding for TFPI mut3EA (5 μg) and carrier DNA (50 μg) of herring sperm DNA) were added to the frozen cells. The cells were then thawed by shaking at 37° C. for 5 min. After addition of 1.5 ml of solution B (40% PEG 1000; 200 mM bicine pH 8.35), the cells were incubated at 30° C. for 60 min and, after pelleting, washed with 1.5 ml of solution C (0.15 M NaCl; 10 mM bicine pH 8.35) and resuspended in 100 μl of solution C. Plating out took place on a selection medium with 2% agar. Transformands were obtained after incubation at 30° C. for 3 days.

### Example 3

### Preparation of TFPI mut3EA by Fermentation of the Yeast Cells

### Nutrient Solutions

The following nutrient solutions were used for fermentation of yeast cells to express TFPI mut3EA:

| | Nutrient solution | | | |
|---|---|---|---|---|
| Ingredient | SD2 | | SC5 | |
| Bacto-yeast nitrogen base | 6.7 | g/l | — | |
| Difco bacto-yeast extract | — | | 20.0 | g/l |
| Glucose | 20.0 | g/l | 20.0 | g/l |
| KH₂PO₄ | 6.7 | g/l | 6.7 | g/l |

| | | | |
|---|---|---|---|
| (NH₄)₂SO₄ | — | 2.0 | g/l |
| MgSO₄ × 7 H₂O | — | 1.0 | g/l |
| Trace element solution 4 | — | 1.0 | ml/l |

| | | |
|---|---|---|
| pH after NaOH titr. | 6 | 6 |

### Trace Element Solution 4 (SL4 Solution):

| | | | |
|---|---|---|---|
| Titriplex III | (Merck 8418) | 5 | g |
| FeSO₄•7H₂O | (Merck 3965) | 2 | g |
| ZnSO₄•7H₂O | (Merck 8883) | 0.1 | g |
| MnCl₂•4H₂O | (Merck 5927) | 30 | mg |
| H₃BO₃ | (Merck 165) | 0.3 | g |
| CoCl₂•6H₂O | (Merck 2533) | 0.2 | g |
| CuCl₂•2H₂O | (Merck 2733) | 10 | mg |
| NiCl₂•6H₂O | (Merck 6717) | 20 | mg |
| Na₂MoO₄•2H₂O | (Merck 6521) | 30 | mg |

The ingredients of the SL4 solution were dissolved in demineralized water and the pH was adjusted to pH 3-4 with NaOH. The nutrient solution was made up to 1000 ml with demineralized water and stored in aliquots at −20° C.

The ingredients of nutrient solutions SD2 and SC5 were made up in demineralized water and the pH was adjusted to pH 5.5. Sterilization took place at 121° C. for 20 min. Glucose was dissolved in ⅕ of the required volume in demineralized water, sterilized separately and, after cooling, added to the remaining nutrient solution.

### Strain Stocks

Strain stocks of all the yeast transformands were set up by mixing 1 ml aliquots of a preculture with 1 ml of 80% glycerol solution and storing at −140° C.

### Precultures

The preculture fermentations were carried out in 50 ml (for small-volume main cultures) or 1 litre shaken flasks (for intermediate-volume main cultures), charged with respectively 10 or 100 ml of SD2 nutrient solution. Inoculation took place with a strain stock or with a single colony from an SD2 agar plate. The cultures were incubated with continuous shaking (240 rpm) at 28-30° C. for 2-3 days.

### Main Culture Fermentations

The main culture fermentations on a small scale took place with use of 1 litre shaken flask charged with 100 ml of SC5 nutrient solution. Inoculation usually took place with 3 ml of the preculture described above. The cultures were then incubated with continuous shaking (240 rpm) at 28-30° C. for 4 days.

In the case of the main culture fermentations on the intermediate scale, the bioreactor system from Wave Biotech (Tagelswangen, CH) was employed. Specifically, 1000 ml of SC5 medium were inoculated with 30 ml of preculture and incubated in a Wavebag with a rocking rate of 32/min for 4 days (angle: 10°; air supply: 0.25 litre/min). The pH of the cultures was monitored on day 1 to 3 and adjusted to pH 5-6 if necessary with 5 M NaOH. On day 1 to 3, 1 ml of 50% strength yeast extract solution and 4 ml of 4 M glucose solution were added to each of the 100 ml cultures.

In the case of the 1000 ml cultures, appropriately 10 or 40 ml of the nutrient solutions were added continuously over the day. To monitor the growth, the OD_{600 nm} of the cultures was determined at various times.

After 4 days, the cell-free supernatants were harvested by centrifugation (15 min at 6000 rpm in a JA14 rotor).

### Example 4

### Purification of TFPI mut3EA from the Supernatants of Fermented Yeast Cells

1 M NaOH was added to the TFPI mut3EA-containing cell-free supernatants prepared in the main culture fermentation until the pH was 7.8. Suspended particles present in the supernatant were sedimented by centrifugation at 2000 rpm at 4° C. (15 min; Beckman-Allegra 6KR). The supernatant was loaded at 1 ml/min onto a 10 ml trypsin-agarose column (Sigma-T1763). The column was then washed with 70 ml of 50 mM Tris pH 7.8, 250 mM NaCl and with 50 ml of 50 mM Tris pH 7.8, 600 mM NaCl. TFPI mut3EA was then eluted with 180 ml of 50 mM KCl/10 mM HCl pH 2.0. The 2 ml fractions were collected in tubes which each contained 500 μl of 200 mM Tris pH 7.6, 2 M NaCl for neutralization. TFPI mut3EA-containing fractions were identified via the inhibition of trypsin in the assay described below.

Trypsin-inhibiting fractions were pooled and dialysed in a dialysis tube with a cutoff of 1000 daltons (Spectra/POR6) twice against 2 litres of 50 mM Tris pH 7.5 each time. The dialysate was concentrated through an ultrafiltration membrane with a cutoff of 1000 daltons in an Amicon 8200 stirred cell. The protein concentration was then determined using a Coomassie plus test (Pierce, 23236) as stated by the manufacturer. The measured protein concentration was typically between 0.1 and 6 mg/ml.

Alternatively, the trypsin-inhibiting fractions were pooled after the purification on trypsin-agarose and mixed with the same volume of 0.1% TFA, and loaded onto a Source 15 RPC column. The column was washed with 6 ml of 0.1% TFA (HPLC-A buffer) and then TFPI mut3EA was eluted with a 25 ml gradient to 50% HPLC-B buffer (0.1% TFA, 60% acetonitrile) and with a further 5 ml gradient to 100% HPLC-B buffer. The TFPI mut3EA-containing eluates were lyophilized and the lyophilizate was taken up in 250 μl of 50 mM Tris pH 7.5 per fraction.

### Example 5

### Determination of the Inhibitory Potency of TFPI mut3EA for Trypsin, Plasmin and Plasma Kallikrein

The inhibitory potency of TFPI mut3 on the enzymatic activities of trypsin, plasmin and plasma kallikrein were determined with the aid of fluorogenic substrates in biochemical assays in white 384-well microtitre plates. The assay buffer was composed of 50 mM Tris/Cl, pH 7.4, 100 mM NaCl, 5 mM CaCl₂, 0.08% (w/v) BSA. The assay conditions were specifically as follows:

| | Final | | | | Final | |
|---|---|---|---|---|---|---|
| | enzyme | | Order | | substrate | Order |
| Enzyme | conc. | | No. | Substrate | conc. | No. |
| Trypsin | 5 | ng/ml | T-6424 | Boc-Ile- | 5 μM | I-1100 |
| | | | (Sigma) | Glu-Gly- | | (Bachem) |
| | | | | Arg-AMC | | |
| Plasmin | 50 | ng/ml | Hplas | MeOSuc- | 50 μM | I-1275 |
| | | | (Enzyme | Ala-Phe- | | (Bachem) |
| | | | Research) | Lys-AMC | | |
| Plasma | 1 | nM | 420307 | Boc-Val- | 80 μM | ES-011 |
| kallikrein | | | (Calbiochem) | Pro-Arg- | | (R&D) |
| | | | | AMC | | |

10 μl of a serial dilution of TFPI mut3EA were introduced into each well and preincubated with 20 μl of enzyme at RT for 5 min. The reaction was then started by adding 20 μl of substrate to each. The measurement took place after 60-90 min in a Tecan reader with an excitation wavelength of 360 nm and an emission wavelength of 465 nm. Dose-activity plots and half-maximum inhibitory constants (IC50 values) were determined using GraphPad Prism software (version 4.02).

### Example 6

### Inhibition of Fibrinolysis by hTFPI D1 Variants

The effect of the hTFPI D1 variants was tested in an in vitro fibrinolysis model and compared with the effect of Trasylol (aprotinin). Human citrated plasma was mixed with the 0.13 μM tissue factor (TF) and 164 U/ml tissue plasminogen activator (tPA) and with hTFPI D1 variants or aprotinin in various concentrations (0.06 μM to 15 μM) and incubated at 37° C. for 40 min. Physiological saline was used as control. The clot formation by TF and the subsequent clot lysis by tPA was determined by measurements of the optical density (OD₄₀₅ nm) with a Tecan Safire. The area under the curve resulting therefrom (AUC) was calculated and plotted against the concentrations of the substances. A reduction in the AUC means inhibition of fibrinolysis. In later experiments the fibrinolysis was defined as relative decrease in the OD after clot formation. The inhibition of the relative decrease in the OD is the measure of the antifibrinolytic activity. The antifibrinolytic activity of TFPI mut3EA and TFPI mut4EA was comparable to that of Trasylol.

### Example 7

### Inhibition of Coagulation by hTFPI D1 Variants

The effect of hTFPI D1 variants was tested in an in vitro coagulation model and compared with the effect of Trasylol (aprotinin). Human citrated plasma was mixed with 12 mM CaCl₂ to induce coagulation and with the hTFPI D1 variants or aprotinin in various concentrations (0.1 μM to 25 μM). Physiological saline was used as control. The OD at 405 nm was determined as a measure of the coagulation during the incubation at 37° C. for 90 min. The half-maximum coagulation time was calculated therefrom. A prolongation of the half-maximum coagulation time means inhibition of coagulation. By comparison with Trasylol, the anticoagulant activity of TFPI mut3EA was increased and that of TFPI mut4EA was slightly increased.

### Example 8

### Haemostatic Effect of hTFPI D1 Variants in a Rat Bleeding Model

Both jugular veins of anaesthetized rats were cannulated with a polyethylene catheter. To prolong the normal bleeding time, the rats were infused with tPA (tissue plasmin activator) (8 mg/kg/h) throughout the experiment. 10 minutes after starting the tPA infusion, the animals were treated with the hTFPI D1 variants or Trasylol (aprotinin) by infusion or with combined bolus administration and subsequent maintenance infusion. In the first experiment, TFPI mut3EA or Trasylol were administered in a dose of 6 mg/kg/h by continuous infusion. In the second experiment, the animals were treated with TFPI mut4EA or Trasylol in doses of 1.5 mg/kg (bolus) and 3 mg/kg/h (infusion) up to 5 mg/kg and 10 mg/kg/h. Physiological saline was used as control in both experiments. 15 minutes after starting the infusion, a transection of the tail (2 mm) was performed, the tip of the tail was immersed in physiological saline at 37° C., and the bleeding time was determined. The bleeding time was defined as the time interval between transection and the visible end of bleeding. Shortening of the bleeding time was the measure of the haemostatic effect. The haemostatic effect of TFPI mut3EA and TFPI mut4EA was comparable to that of Trasylol.

### Example 9

### Antithrombotic Effect of TFPI mut4EA in a Rat Arteriovenous (AV) Shunt Model

The antithrombotic effect of TFPI mut4EA was investigated in a rat arteriovenous shunt model. The carotid artery and the jugular vein of anaesthetized rats were cannulated with a polyethylene catheter, and the catheters were connected together by a small piece of tubing (shunt). A roughened nylon loop was introduced into the tubing and served as thrombogenic surface. Thrombus formation was followed after extracorporeal circulation of the blood through the shunt for 15 minutes. The resulting thrombus was then removed from the tubing, and the thrombus weight was determined. The rats were treated with TFPI mut4EA or Trasylol (aprotinin) by bolus administration and subsequent maintenance infusion. The doses employed were 0.15 mg/kg (bolus) and 0.3 mg/kg/h (infusion) up to 5 mg/kg and 10 mg/kg/h. Physiological saline was used as control. The reduction in the thrombus weight was the measure of the antithrombotic effect. TFPI mut4EA showed an antithrombotic effect comparable to that of Trasylol.

### Example 10

### Influence of Trasylol and TFPI mut4EA on the Relative Mesenteric Bleeding Time in Rats

Wistar rats (250-300 g) were anaesthetized intraperitoneally (thiopental Na, 100 mg/kg) and provided with a vein catheter. After opening the abdomen, a loop of small intestine was transferred outside. While irrigating with warm saline solution, a small mesenteric artery was severed with the aid of microscissors under a stereo-microscope. The bleeding time of three control cuts before administration of the substance and of one cut after administration of the test substance was measured. The bleeding time after treatment with the substance was related to the control bleeding time before administration of the substance in each animal individually.

Trasylol reduced the mesenteric bleeding time dose-dependently (FIG. 11). Administration of 1.5 mg/kg Trasylol as bolus with subsequent infusion (3 mg/kg/h) had no significant effects on the bleeding time. A higher dose of 5 mg/kg as bolus with 10 or 30 mg/kg/h as infusion reduced the bleeding time correspondingly by 28.5±7.4% and 42.7±4.2%.

TFPI mut4EA (FIG. 12) reduced the mesenteric bleeding time in a similar manner to Trasylol. Administration of 5 mg/kg TFPI mut4EA as bolus and 10 mg/kg/h as infusion shortened the bleeding time by 23.6%. The greatest effects of TFPI mut4EA were observed after administration of 5 mg/kg as bolus and 30 mg/kg/h as infusion. At this dose, TFPI mut4EA reduced the bleeding time by 31.7%.

### Example 11

### Investigations of the Effect of hTFPI D1 Variants on the Cardiovascular System in Anaesthetized Rats

The rats were anaesthetized with pentobarbital Na. During the experiment, the rats breathed spontaneously, and the body temperature was kept constant by a heating mat. The arterial blood pressure was measured via a catheter in the carotid artery using a pressure transducer and a pressure measurement bridge. The ECG was recorded with the 3 standard extremity leads by means of an ECG amplifier. The measured signals were acquired, evaluated and stored by a software. The systolic, diastolic and mean blood pressure, and the heart rate, were calculated from the blood pressure signal. The ECG was additionally assessed and evaluated manually after the experiment. There was additionally analogue recording of the blood pressure and ECG signals.

Following installation of the catheters and a course phase (constant blood pressure and heart rate), TFPI mut3EA or TFPI mut4EA was administered i.v. as bolus injection or continuous infusion. After a fixed observation period, the experiment was terminated by sacrificing the animals by an anaesthetic overdose.

### Example 12

### Inhibition of the IL-8- and C5a-Induced Chemotaxis of Neutrophils by hTFPI D1 Variants

Neutrophils were isolated from blood by standard methods. Chemotaxis of the neutrophils was carried out in a two-chamber system. An HUVEC monolayer was cultured on the membrane used (3-μm pore size, polycarbonates, from Falcon) for 24 h.

1×10⁵ neutrophils in RPMI 1640 medium, which had previously been loaded with a fluorescent dye, were put into the upper chamber. The lower chamber contained varying concentrations of stimulus or a constant stimulus concentration (IL-8: 5 nM or C5a: 10 nM) and varying concentrations of the test substances Trasylol (aprotinin), TFPI mut3EA or TFPI mut4EA. The substances to be investigated were present in both chambers. The test mixture was incubated at 37° C. and 5% CO₂ for 45 min. After the incubation, the cells which had migrated into the lower chamber were determined (fluorescence measurement, counting).

### Example 13

### Inhibition of the CAP-37-Induced Increase in Permeability by hTFPI D1 Variants

A confluent monolayer of HUVEC was cultured on the membrane of the insert in a two-chamber system (Falcon). For this purpose, 2×10⁵ cells were seeded per insert and incubated (37° C./5% CO₂) for 18-20 h. The continuity of the monolayer was checked by means of a trypan blue-conjugated albumin solution before starting the experiment. Then CAP37 (5 μM) was put in the upper chamber. The change in permeability was determined in the presence of varying concentrations (10 μM-0.01 μM) of the test substances Trasylol (aprotinin), TFPI mut3EA or TFPI mut4EA for 3 h. The efflux of the trypan blue-conjugated albumin solution served in this case as indicator of the change in permeability. The OD is measured at 590 nm.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### REFERENCES

Apeler et al. [2004] Apeler, H. et al. (2004). Arzneimittelforschung 54, 483-497.
Apeler [2005] Apeler, H. in: J. Knäblein (Ed.), Modern Biopharmaceuticals, WILEY-VHC, Weinheim, 2005, pp. 1021-1032
Bajaj et al. [2001] Bajaj, M. S. (2001). Thromb Haemost 86, 959-972.
Beierlein et al. [2005] Beierlein, W. et al. (2005). Ann Thorac Surg 79, 741-748.
Blauhut et al. [1991] Blauhut, B. et al. (1991). J Thorac Cardiovasc Surg 101, 958-967.
Bode und Huber. [1992] Bode, W., und Huber, R. (1992). Eur J Biochem 204, 433-451.
Broze et al. [1987] Broze, G. J. et al. (1987). Thromb Res 48, 253-259.
Dennis et al. [1995] Dennis, M. S. et al. (1995). J Biol Chem 270, 25411-25417.
Dietrich et al. [2001] Dietrich, W. et al. (2001). Anesthesiology 95, 64-71.
Dietrich et al. [1995] Dietrich, W. et al. (1995). Anesthesiology 83, 679-689.
Fritz und Wunderer [1983] Fritz, H., und Wunderer, G. (1983). Arzneimittelforschung 33, 479-494.
Girard et al. [1989] Girard, T. J. et al. (1989). Nature 388, 518-520.
Hess [2005] Hess, P. J. Jr (2005). Am J Health Syst Pharm 62 (18Suppl 4), 6-9.
Krowarsch et al. [2005] Krowarsch, D. et al. (2005). Protein Pept Lett 12, 403-407.
Laskowski und Kato [1980] Laskowski, M., und Kato, 1. (1980). Ann Rev Biochem 49, 593-626.
Lindahl [1997] Lindahl, A. K. (1997). Cardiovasc Res 33, 286-291.
Lwaleed und Bass [2005] Lwaleed, B. A., und Bass, P. S. (2005). J Pathol, DOI:
Markland et al. [1996a] Markland, W. et al. (1996). Biochemistry 35, 8045-8057.
Markland et al. [1996b] Markland, W. et al. (1996). Biochemistry 35, 8058-8067.
Novotny et al. [1989] Novotny, W. F. et al. (1989). J Biol Chem 264, 18832-18837.
Otlewski et al. [2001] Otlewski, J. et al. (2001). Acta Biochim Pol 48, 419-428.
Petersen et al. [1996] Petersen, L. C. et al. (1996). Eur J Biochem 235, 310-316.
Royston [1992] Royston, D. (1992). J Cardiothorac Vase Anesth 6, 76-100.
Schechter und Berger [1967] Schechter, I., und Berger, A. (1967). Biochem. Biophys. Res. Commun. 27, 157-162.
Sprecher et al. [1994] Sprecher, C. A. (1994). Proc Natl Acad Sci USA 91, 3353-3357.
Wun et al. [1988] Wun, T.-C. et al. (1988). J Biol Chem 263, 6001-6004.

### ABBREVIATIONS

AUC area under the curve
BPTI bovine pancreatic trypsin inhibitor
DIC disseminated intravascular coagulation
DNA deoxyribonucleic acid
ECG electrocardiogram
HepG2 human hepatoma cell line
HPLC high performance liquid chromatography
hTFPI human tissue factor pathway inhibitor 1
hTFPI D1 human tissue factor pathway inhibitor 1 Kunitz domain 1
HUVEC human umbilical vein endothelial cells
IC50 inhibitor concentration at 50% inhibition of enzymic activity
IL-8 interleukin 8
i.v. intravenous

### kDa Kilodalton

LACI lipoprotein-associated coagulation inhibitor
M molar (mol per litre)

### Min Minute

MOF multiorgan failure

### OD Absorption

PEG polyethylene glycol
PCR polymerase chain reaction
Rpm revolutions per minute
TF tissue factor
TFA trifluoroacetic acid
TPA tissue plasminogen activator
U unit (unit of enzymic activity)

TABLE 1
Definition of a Kunitz domain

1 2 3 4 5
1234567890123456789012345678901234567890123456789012345678
xxxxCxxxxxxGxCxxxxxxXXXxxxxxxCxxFxXXGCxXxxXxXxxxxxCxxxCxxx

X21 = Phe, Tyr, Trp
X22 = Tyr, Phe, Trp, Ala, His
X23 = Tyr or Phe
X35 = Tyr, Phe, Ser
X36 = Gly, Ser, Arg, Thr
X40 = Gly, Ala, Arg
X43 = Asn or Gly
X45 = Phe or Tyr
Amino acid 1, 2, 3, 4 may be absent
Amino acid 56, 57, 58 may be absent

TABLE 2
Sequences of some natural Kunitz domains

| | NCBI Acces- | |
|---|---|---|
| Protein | sion No. | Sequence |
| Human amyloid beta A4 | P05067 | EVCSEQAETGPCRAMISRWYFDVTEGKCAPFFYGGCGGNRNNFDTEEYCMAVCGSA |
| protein precursor; AA 289-344 | | SEQ ID NO: 41 |
| Aprotinin; pancreatic trypsin | NP_001001554 | RPDFCLEPPYTGPCKARIIRYFYNAKAGLCQTFVYGGCRAKRNNFKSAEDCMRTCGGA |
| inhibitor precursor; AA36-94 | | SEQ ID NO: 42 |
| Human eppin precursor; | O95925 | KQDVCEMPKETGPCLAYFLHWWYDKKDNTCSMFVYGGCQGNNNNFQSKANCLNTC |
| AA 73-127 | | SEQ ID NO: 43 |
| Human placental bikunin; | O43291 | DFCLVSKVVGRCRASMPRWWYNVTDGSCQLFVYGGCDGNSNNYLTKEECLKKC |
| AA 36-38 (domain 1) | | SEQ ID NO: 44 |
| Human placental bikunin; | O43291 | EYCTANAVTGPCRASFPRWYFDVERNSCNNFIYGGCRGNKNSYRSEEACMLRC |
| AA 131-183 (domain 1) | | SEQ ID NO: 45 |
| Human amyloid-like protein 2 | Q06481 | CSQEAMTGPCRAVMPRWYFDLSKGKCVRFIYGGCGGNRNNFESEDYCMAVC |
| precursor; AA310-360 | | SEQ ID NO: 46 |
| WFIKKN; AA 385-437 | Q8TEU8 | PLAACSLPALQGPCKAYAPRWAYNSQTGQCQSFVYGGCEGNGNNFESREACEESCPF |
| (domain 2) | | SEQ ID NO: 47 |

TABLE 3
Sequences of some non-natural Kunitz domains

| | | |
|---|---|---|
| Protein | Reference | Sequence |

| | | |
|---|---|---|
| Aprotinin variant | Apeler et al. | R- |
| (DesPro2-Ser10-Arg 15- | [2004] | DPCLEPPSTGPCRAAIIRYFYDATAGLCETFVYGGCRANRNNFKSAEDCMETCGGA |
| Ala 17-Asp24-Thr26-Glu31- | | SEQ ID NO: 48 |
| Asn41-Glu53) | | |
| Aprotinin variant | EP 0307592 | RPDFCLEPPYTGPCKAAIIRYFYNAKAGLCQTFVYGGCRAKRNNFKSAEDCERTCGGA |
| (Ala17-Glu52) | | SEQ ID NO: 49 |
| Aprotinin variant | EP 0238993 | RPDFCLEPPYTGPCRARIIRYFYNAKAGLCQTFVYGGCRAKRNNFKSAEDCERTCGGA |
| (Arg15-Glu52) | | SEQ ID NO: 50 |
| KALI-DY | Dennis et al. | EVCSEQAEDGHCRAAHPRWYFDVTEGKCAPFVYGGCGGNRNNFDTEEYCMAVCGSA |
| | [1995] | SEQ ID NO: 51 |

TABLE 4
IC50 values of some Kunitz domains for inhibition
of human trypsin, plasmin and plasma kallikrein

| | Trypsin | Plasmin | Plasma kallikrein |
|---|---|---|---|
| Kunitz domain | IC50 [nM] | IC50 [nM] | IC50 [nM] |
| Aprotinin (BPTI) | 9 | 4 | 46 |
| hTFPI D1 | 44 | 559 | >10.000 |
| SEQ ID NO: 4 | 5 | 32 | 3 |
| SEQ ID NO: 5 | 8 | 24 | 3 |
| SEQ ID NO: 7 | 19 | 195 | 11 |
| SEQ ID NO: 8 | 266 | 267 | 1 |
| SEQ ID NO: 9 | 36 | 497 | >10.000 |
| SEQ ID NO: 10 | 6 | 27 | 48 |
| SEQ ID NO: 18 | 20 | 47 | 1.480 |
| SEQ ID NO: 19 | 5 | 30 | 3. |
| SEQ ID NO: 20 | 7 | 24 | 2 |
| SEQ ID NO: 25 | 38 | 52 | 1.186 |
| SEQ ID NO: 26 | 64 | 83 | 194 |
| SEQ ID NO: 28 | 17 | 77 | 8 |
| SEQ ID NO: 29 | 4 | 19 | 16 |
| SEQ ID NO: 30 | 21 | 56 | 2.336 |
| SEQ ID NO: 31 | 1 | 48 | 75 |

TABLE 5
Amino acids from the region with inhibitory activity of natural or non-natural Kunitz
domains which can be replaced by the corresponding amino acids from hTFPI D1

| | NCBI accession No. | | | | | | | | | | |
|---|---|---|---|---|---|---|---|---|---|---|---|
| Kunitz domain | or reference | X₁₀ | X₁₁ | X₁₅ | X₁₆ | X₁₇ | X₁₈ | X₁₉ | X₂₀ | X₂₁ | X₂₂ |
| Human amyloid beta A4 protein | P05067 | E | T | | | | | | | | |
| precursor; AA 289-344 | | | | | | | | | | | |
| Aprotinin; pancreatic trypsin | NP_001001554 | Y | T | | | R | I | I | | | |
| inhibitor precursor; AS 36-94 | | | | | | | | | | | |
| Human eppin precursor; | O95925 | E | T | | | | | | | | |
| AA 73-127 | | | | | | | | | | | |
| Human placental bikunin; | O43291 | | | R | | | | P | | W | W |
| AA 36-88 (domain 1) | | | | | | | | | | | |
| Human placental bikunin; | O43291 | V | T | R | | S | F | P | | W | |
| AA 131-183 (domain 2) | | | | | | | | | | | |
| Human amyloid-like protein 2 | Q06481 | | | | | | | P | | W | |
| precursor; AA 310-360 | | | | | | | | | | | |
| WFIKKN; AS 385-437 (domain 2) | Q8TEU8 | | | | | | | P | | W | |
| Aprotinin variant (DesPro2-Ser10-Arg15- | Apeler et al. [2004] | S | T | R | | A | I | I | | | |
| Ala17-Asp24-Thr26-Glu31-Asn41-Glu53) | | | | | | | | | | | |
| Aprotinin variant (Ala17-Glu52) | EP 0307592 | Y | T | | | A | I | I | | | |
| Aprotinin variant (Arg15-Glu52) | EP 0238993 | Y | T | R | | R | I | I | | | |
| KALI-DY | Dennis et al. [1995] | | | R | | A | H | P | | W | |
| hTFPI D1 | P10646 | D | D | K | A | I | M | K | R | F | F |

1. An isolated hTFPI D1 variant of the general formula:
MHSFCAFKAX₁₀X₁₁GPCX₁₅X₁₆X₁₇X₁₈X₁₉X₂₀X₂₁X₂₂FNIFTRQCEEFIYGGCEGNQNRF ESLEECKKMCTRD
having at least one mutation in positions X₁₀, X₁₁, X₁₅, X₁₆, X₁₇, X₁₈, X₁₉, X₂₀, X₂₁ and X₂₂ compared with the natural sequence of SEQ ID NO: 2, wherein:
X₁₀ is the amino acids D, E, Y, V or S;
X₁₁ is the amino acids D or T;
X₁₅ is the amino acids K, or R;
X₁₆ is the amino acids A;
X₁₇ is the amino acids I, R, S or A;
X₁₈ is the amino acids M, I, F or H;
X₁₉ is the amino acids K, I or P;
X₂₀ is the amino acids R;
X₂₁ is the amino acids F or W; and
X₂₂ is the amino acids F or W,
excluding the peptide of the sequence
SEQ ID No. 1 (TFPI mut1).

2. An isolated hTFPI D1 variant according to claim 1, wherein the variant additionally comprises the amino acids E and A at the N terminus.

3. An isolated hTFPI D1 variant according to claim 1 selected from the group comprising: SEQ ID NO. 3 (TFPI mut13), SEQ ID NO. 4 (TFPI mut3), SEQ ID NO. 5 (TFPI mut4), SEQ ID NO. 7 (TFPI mut2), SEQ ID NO. 8 (TFPI mut5), SEQ ID NO. 9 (TFPI mut6), SEQ ID NO. 10 (TFPI random), SEQ ID NO. 11 (TFPI mut7), SEQ ID NO. 12 (TFPI mut8), SEQ ID NO. 13 (TFPI mut9), SEQ ID NO. 14 (TFPI mut10), SEQ ID NO. 15 (TFPI mut11), and SEQ ID NO. 16 (TFPI mut12).

4. An isolated hTFPI D1 variant according to claim 2 selected from the group comprising: SEQ ID NO. 17 (TFPI mut15), SEQ ID NO. 18 (TFPI mut13EA), SEQ ID NO. 19 (TFPI mut3EA), SEQ ID NO. 20 (TFPI mut4EA), SEQ NO. 21 (TFPI mut2EA), SEQ ID NO. 22 (TFPI mut5EA), SEQ ID NO. 23 (TFPI mut6EA), SEQ ID NO. 24 (TFPI randomEA), SEQ ID NO. 25 (TFPI mut7EA), SEQ ID NO. 26 (TFPI mut8EA), SEQ ID NO. 27 (TFPI mut9EA), SEQ ID NO. 28 (TFPI mut10EA), SEQ ID NO. 29 (TFPI mut11EA), SEQ ID NO. 30 (TFPI mut12EA), and SEQ ID NO. 31 (TFPI mut15EA).

5. An isolated DNA sequence coding for an isolated hTFPI D1 variant according to claim 1.

6. An expression vector comprising the isolated DNA sequence according to claim 5 and a promoter operably linked to the DNA sequence.

7. A microorganism comprising the isolated DNA sequence according to claim 5.

8. A method for preparing an isolated hTFPI D1 variant according to claim 1 by expression of the expression vector according to claim 6.

9. A pharmaceutical composition comprising a pharmaceutically acceptable amount of an isolated hTFPI D1 variant of claim 1 and a pharmaceutically acceptable carrier.


SEQUENCE LISTING (53 sequences)

SEQ ID NO: 1 - protein, 58 aa, Homo sapiens
MHSFCAFKADDGPCRAAHPRWFFNIFTRQCEEFIYGGCEGNQNRFESLEECKKMCTRD

SEQ ID NO: 2 - protein, 58 aa, Homo sapiens
MHSFCAFKADDGPCKAIMKRFFFNIFTRQCEEFIYGGCEGNQNRFESLEECKKMCTRD

SEQ ID NO: 3 - protein, 58 aa, Homo sapiens
MHSFCAFKAVTGPCKARIIRFFFNIFTRQCEEFIYGGCEGNQNRFESLEECKKMCTRD

SEQ ID NO: 4 - protein, 58 aa, Homo sapiens
MHSFCAFKAETGPCRAAHPRWFFNIFTRQCEEFIYGGCEGNQNRFESLEECKKMCTRD

SEQ ID NO: 5 - protein, 58 aa, Homo sapiens
MHSFCAFKAETGPCRAAHPRWWFNIFTRQCEEFIYGGCEGNQNRFESLEECKKMCTRD

SEQ ID NO: 6 - protein, 58 aa, Bovine [sequence omitted]

SEQ ID NO: 7 - protein, 58 aa, Homo sapiens
MHSFCAFKADDGPCRAAMKRFFFNIFTRQCEEFIYGGCEGNQNRFESLEECKKMCTRD

SEQ ID NO: 8 - protein, 58 aa, Homo sapiens
MHSFCAFKADDGPCRAAHPRWWFNIFTRQCEEFIYGGCEGNQNRFESLEECKKMCTRD

SEQ ID NO: 9 - protein, 58 aa, Homo sapiens
MHSFCAFKADDGPCKAIMKRFWFNIFTRQCEEFIYGGCEGNQNRFESLEECKKMCTRD

SEQ ID NO: 10 - protein, 58 aa, Homo sapiens
MHSFCAFKAETGPCRAAMKRFFFNIFTRQCEEFIYGGCEGNQNRFESLEECKKMCTRD

SEQ ID NO: 11 - protein, 58 aa, Homo sapiens
MHSFCAFKAYTGPCKARIIRFFFNIFTRQCEEFIYGGCEGNQNRFESLEECKKMCTRD

SEQ ID NO: 12 - protein, 58 aa, Homo sapiens
MHSFCAFKAYTGPCKAAIIRFFFNIFTRQCEEFIYGGCEGNQNRFESLEECKKMCTRD

SEQ ID NO: 13 - protein, 58 aa, Homo sapiens
MHSFCAFKAYTGPCRARIIRFFFNIFTRQCEEFIYGGCEGNQNRFESLEECKKMCTRD

SEQ ID NO: 14 - protein, 58 aa, Homo sapiens
MHSFCAFKAYTGPCRAAIIRFFFNIFTRQCEEFIYGGCEGNQNRFESLEECKKMCTRD

SEQ ID NO: 15 - protein, 58 aa, Homo sapiens
MHSFCAFKAVTGPCRASFPRWFFNIFTRQCEEFIYGGCEGNQNRFESLEECKKMCTRD

SEQ ID NO: 16 - protein, 58 aa, Homo sapiens
MHSFCAFKASTGPCKARIIRFFFNIFTRQCEEFIYGGCEGNQNRFESLEECKKMCTRD

SEQ ID NO: 17 - protein, 58 aa, Homo sapiens
MHSFCAFKAVTGPCRAAMKRFWFNIFTRQCEEFIYGGCEGNQNRFESLEECKKMCTRD

SEQ ID NO: 18 - protein, 60 aa, Homo sapiens
EAMHSFCAFKAVTGPCKARIIRFFFNIFTRQCEEFIYGGCEGNQNRFESLEECKKMCTRD

SEQ ID NO: 19 - protein, 60 aa, Homo sapiens
EAMHSFCAFKAETGPCRAAHPRWFFNIFTRQCEEFIYGGCEGNQNRFESLEECKKMCTRD

SEQ ID NO: 20 - protein, 60 aa, Homo sapiens
EAMHSFCAFKAETGPCRAAHPRWWFNIFTRQCEEFIYGGCEGNQNRFESLEECKKMCTRD

SEQ ID NO: 21 - protein, 60 aa, Homo sapiens
EAMHSFCAFKADDGPCRAAMKRFFFNIFTRQCEEFIYGGCEGNQNRFESLEECKKMCTRD

SEQ ID NO: 22 - protein, 60 aa, Homo sapiens
EAMHSFCAFKADDGPCRAAHPRWWFNIFTRQCEEFIYGGCEGNQNRFESLEECKKMCTRD

SEQ ID NO: 23 - protein, 60 aa, Homo sapiens
EAMHSFCAFKADDGPCKAIMKRFWFNIFTRQCEEFIYGGCEGNQNRFESLEECKKMCTRD

SEQ ID NO: 24 - protein, 60 aa, Homo sapiens
EAMHSFCAFKAETGPCRAAMKRFFFNIFTRQCEEFIYGGCEGNQNRFESLEECKKMCTRD

SEQ ID NO: 25 - protein, 60 aa, Homo sapiens
EAMHSFCAFKAYTGPCKARIIRFFFNIFTRQCEEFIYGGCEGNQNRFESLEECKKMCTRD

SEQ ID NO: 26 - protein, 60 aa, Homo sapiens
EAMHSFCAFKAYTGPCKAAIIRFFFNIFTRQCEEFIYGGCEGNQNRFESLEECKKMCTRD

SEQ ID NO: 27 - protein, 60 aa, Homo sapiens
EAMHSFCAFKAYTGPCRARIIRFFFNIFTRQCEEFIYGGCEGNQNRFESLEECKKMCTRD

SEQ ID NO: 28 - protein, 60 aa, Homo sapiens
EAMHSFCAFKAYTGPCRAAIIRFFFNIFTRQCEEFIYGGCEGNQNRFESLEECKKMCTRD

SEQ ID NO: 29 - protein, 60 aa, Homo sapiens
EAMHSFCAFKAVTGPCRASFPRWFFNIFTRQCEEFIYGGCEGNQNRFESLEECKKMCTRD

SEQ ID NO: 30 - protein, 60 aa, Homo sapiens
EAMHSFCAFKASTGPCKARIIRFFFNIFTRQCEEFIYGGCEGNQNRFESLEECKKMCTRD

SEQ ID NO: 31 - protein, 60 aa, Homo sapiens
EAMHSFCAFKAVTGPCRAAMKRFWFNIFTRQCEEFIYGGCEGNQNRFESLEECKKMCTRD

SEQ ID NO: 32 - protein, 58 aa, Homo sapiens
  misc_feature: X = natural amino acid
MHSFCAFKAXXGPCXXXXXXXXFNIFTRQCEEFIYGGCEGNQNRFESLEECKKMCTRD

SEQ ID NO: 33 - protein, 60 aa, Homo sapiens
  misc_feature: X = natural amino acid
EAMHSFCAFKAXXGPCXXXXXXXXFNIFTRQCEEFIYGGCEGNQNRFESLEECKKMCTRD

SEQ ID NO: 34 - DNA, 48 bp, Homo sapiens
ggtaagcttggataaaagagaagctatgcattttttttgtgcttttaa

SEQ ID NO: 35 - DNA, 41 bp, Homo sapiens
tagtggatcccgagcttgcttattaatctctagtacacatt

SEQ ID NO: 36 - DNA, 88 bp, Homo sapiens
ctatgcattctttttgtgcttttaaagctgatgatggtccatgtagagctgctcatccaagatggtttttcaatatttttacaagaca

SEQ ID NO: 37 - DNA, 30 bp, Homo sapiens
tccctcgagttattaatctctagtacacat

SEQ ID NO: 38 - DNA, 42 bp, Homo sapiens
tttgtgcttttaaagctgatgatggtccatgtagagctgctc

SEQ ID NO: 39 - DNA, 42 bp, Homo sapiens
tttgtgcttttaaagctgaaactggtccatgtagagctgctc

SEQ ID NO: 40 - DNA, 42 bp, Homo sapiens
gagcagctctacatggaccagtttcagctttaaaagcacaaa

SEQ ID NO: 41 - protein, 56 aa, Homo sapiens
EVCSEQAETGPCRAMISRWYFDVTEGKCAPFFYGGCGGNRNNFDTEEYCMAVCGSA

SEQ ID NO: 42 - protein, 58 aa, Homo sapiens [sequence omitted]

SEQ ID NO: 43 - protein, 55 aa, Homo sapiens
KQDVCEMPKETGPCLAYFLHWWYDKKDNTCSMFVYGGCQGNNNNFQSKANCLNTC

SEQ ID NO: 44 - protein, 53 aa, Homo sapiens
DFCLVSKVVGRCRASMPRWWYNVTDGSCQLFVYGGCDGNSNNYLTKEECLKKC

SEQ ID NO: 45 - protein, 53 aa, Homo sapiens
EYCTANAVTGPCRASFPRWYFDVERNSCNNFIYGGCRGNKNSYRSEEACMLRC

SEQ ID NO: 46 - protein, 51 aa, Homo sapiens
CSQEAMTGPCRAVMPRWYFDLSKGKCVRFIYGGCGGNRNNFESEDYCMAVC

SEQ ID NO: 47 - protein, 57 aa, Homo sapiens
PLAACSLPALQGPCKAYAPRWAYNSQTGQCQSFVYGGCEGNGNNFESREACEESCPF

SEQ ID NO: 48 - protein, 56 aa, Homo sapiens
DFCLEPPSTGPCRAAIIRYFYDATAGLCETFVYGGCRANRNNFKSAEDCMETCGGA

SEQ ID NO: 49 - protein, 58 aa, Homo sapiens
RPDFCLEPPYTGPCKAAIIRYFYNAKAGLCQTFVYGGCRAKRNNFKSAEDCERTCGGA

SEQ ID NO: 50 - protein, 58 aa, Homo sapiens; identical to SEQ ID NO: 49

SEQ ID NO: 51 - protein, 56 aa, Homo sapiens
EVCSEQAEDGHCRAAHPRWYFDVTEGKCAPFVYGGCGGNRNNFDTEEYCMAVCGSA

SEQ ID NO: 52 - DNA, 299 bp, Homo sapiens
tcaaacaagaagattacaaactatcaatttcatacacaatataaacgattaaaagaatgagattcccatctatttttactgctgttttgtttgctgcttcttctgctttggctatgcattctttttgtgcttttaaagctgatgatggtccatgtaaagctattatgaaaagattttttttcaatatttttacaagacaatgtgaagagtttatctatggtggttgtgaaggtaatcaaaatagatttgagtctttggaagagtgtaaaaaaatgtgtactagagattaataactcgag

SEQ ID NO: 53 - DNA, 540 bp, Homo sapiens
ttcaaacaagaagattacaaactatcaatttcatacacaatataaacgattaaaagaatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggataaaagagaagctatgcattctttttgtgcttttaaagctgatgatggtccatgtaaagctattatgaaaagattttttttcaatatttttacaagacaatgtgaagagtttatctatggtggttgtgaaggtaatcaaaatagatttgagtctttggaagagtgtaaaaaaatgtgtactagagattaataagcaagctcgggatccactagtaacggccgccagtgtgctgga